(19) United States
(12) Patent Application Publication
(10) Pub. No.: US 2010/0247664 A1
(43) Pub. Date: Sep. 30, 2010

(54) METHOD OF PREVENTING OR TREATING METABOLIC SYNDROME

(76) Inventors: Stephanie K. Lynch, Brooklyn, NY (US); Wallace Yokoyama, Berkely, CA (US); Maciej Turowski, Midland, MI (US); Jerry R. Conklin, Midland, MI (US)

Correspondence Address: The Dow Chemical Company, P.O. BOX 1967, Midland, MI, 48641, US

(21) Appl. No.: 12/446,371

(22) PCT Filed: Oct. 18, 2007
(86) PCT No.: PCT/US07/81785

§ 371 Date: May 14, 2010

Related U.S. Application Data
(60) Provisional application No. 60/853,389, filed on Oct. 20, 2006.

(51) Int. Cl. A61K 31/717; A61K 9/14; C08B 11/02

(52) U.S. Cl. 424/499; 514/57; 536/100

(57) ABSTRACT

Water-insoluble cellulose derivatives such as ethyl cellulose can be used to treat or prevent metabolic syndrome and/or one of the abnormalities of metabolic syndrome.

This invention was made under a Cooperative Research And Development Agreement with the US Department of Agriculture, number 58-3K95-5-1072.

### FIELD OF THE INVENTION

This invention relates to a method of preventing or treating metabolic syndrome or a symptom or condition associated with the metabolic syndrome and to a medicament, pharmaceutical composition, food, food ingredient or supplement, or nutraceutical ingredient or supplement useful in such method.

### BACKGROUND OF THE INVENTION

Metabolic syndrome is a complex disease, characterized by the American Heart Association by the following abnormalities: abdominal obesity, atherogenic dyslipidemia, hypertension, insulin resistance with or without glucose intolerance, proinflammatory state and prothrombotic state (Grundy et al., “DEFINITION OF METABOLIC SYNDROME” Circulation, 2004, V109, pages 433-438, Document Number DOI: 10.1161/01.CIR.0000111245.75752.C6 available at www.circulationaha.org, herein fully incorporated by reference). It is generally recognized in the art that people with three or more of the above symptoms can be considered to have the metabolic syndrome. The American Heart Association estimates that about 20 to 25 percent of US adults have the metabolic syndrome. People with the metabolic syndrome are at increased risk of a cardiovascular disease, such as coronary heart disease or other diseases related to plaque buildups in artery walls (e.g., stroke and peripheral vascular disease) and/or Type II diabetes. Cardiovascular diseases and type II diabetes belong to the most pervasive diseases in Western populations. Diabetes mellitus is a disease which affects millions people in the United States and, although a heterogeneous disorder, it generally is classified within two major categories, i.e., Type I and Type II diabetes. About 80% of all diabetics in the United States are in the Type II category. This type of diabetes is characterized by both impaired insulin secretion and insulin resistance. The majority of patients are obese adults and loss of weight can restore normoglycemia in some cases. However, this type of diabetes can also occur in the non-obese adults and in children. Evidently there is an urgent need to find a method of preventing or treating metabolic syndrome or a symptom or condition associated with the metabolic syndrome.

Since cardiovascular diseases and type II diabetes belong to the most pervasive diseases in Western populations, huge research efforts are not only spent on finding methods of preventing or treating metabolic syndrome, but also on the diagnosis of the symptoms of the metabolic syndrome including biological markers and on trying to understand the biological processes that influence the various symptoms of the metabolic syndrome.

The above-mentioned article by Grundy et al., “DEFINITION OF METABOLIC SYNDROME”, teaches that a proinflammatory state is recognized clinically by elevations of C-reactive protein (CRP). Multiple mechanisms seemingly underlie elevations or CRP. According to the Online Dictionary MidlinePlus Medical Encyclopedia, CRP is a special type of protein produced by the liver that is only present during episodes of acute inflammation. The Medical Encyclopedia indicates that it is not known whether CRP is merely a marker of disease or whether it actually plays a role in causing artherosclerotic disease, but that many consider elevated CRP to be a positive risk factor for coronary artery disease.

The above-mentioned article by Grundy et al., “DEFINITION OF METABOLIC SYNDROME”, further teaches that a prothrombotic state is characterized by increased Plasminogen Activator Inhibitor-1 (PAI-1) and fibrinogen. Fibrinogen, an acute-phase reactant like CRP, rises in response to a high-cytokine state. Grundy et al. suggest that prothrombotic state and proinflammatory states may be metabolically interconnected.

A. Zambon et al. have published in Biochemical Society Transactions (2003) Volume 31, part 5, page 1070 et seq. the article “Relevance of hepatic lipase to the metabolism of triacylglycerol-rich lipoproteins”. Hepatic lipase (HL) is a glycoprotein that is synthesized and secreted by the liver. HL catalyzes the hydrolysis of triacylglycerols and phospholipids in different lipoproteins. HL may have pro- as well as anti-atherogenic effects. In the presence of hypertriglyceridaemia or an increased LDL (low density lipoproteins) concentration, the pro-atherogenic effect of high HL may prevail. However, among individuals with low levels of LDL, having high levels of HL may not be atherogenic, but rather anti-atherogenic.

In view of the above-discussed impact of C-reactive protein, of Plasminogen Activator Inhibitor-1, and, depending on the individuals, also of hepatic lipase on one or more symptoms of the metabolic syndrome, it would be desirable to find a method of influencing the level of expression or the concentration of C-reactive protein, of Plasminogen Activator Inhibitor-1, of hepatic lipase or of two or three thereof.

In addition to the impact of C-reactive protein, Plasminogen Activator Inhibitor-1, and hepatic lipase on metabolic syndrome, skilled artisans have suggested adiponectin as a key potential player in metabolic syndrome.

Tohru Funahashi, Yuji Matsuzawa and Shinji Kihara, “Adiponectin as a key potential player in metabolic syndrome”, International Congress Series 1262 (2004), Pages 368-371 suggest that hyposecretion of adiponectin may play an important role in the development of obesity-related diseases, particularly atherosclerosis, Diabetes Mellitus, Inflammation and cancer.

Mori Y, Hoshino K, Yokota K, Itoh Y, Tajima N., “Role of hypoadiponectinemia in the metabolic syndrome and its association with post-glucose challenge hyper-free fatty acidemia: a study in prediabetic Japanese males”, Endocrine, 2006 Apr.; 29(2):357-61 suggest that adiponectin is closely associated with the multiple risk factors that go to make up the metabolic syndrome, suggesting a role for hypoadiponectinemia as a surrogate marker for the metabolic syndrome.

Adipocytes express a variety of proteins that function in the homeostatic control of glucose and lipid metabolism. Insulin regulates the translocation and secretion of many of these proteins in response to changes in energy balance. Adipocyte complement-related protein of 30 kDa (Acrp30), now known as adiponectin, is a protein whose secretion from adipocytes is enhanced by insulin stimulation. Adiponectin is an unique and essential adipocytokine that is produced very abundantly in adipocytes and stably present in the plasma at very high concentration (Matsuzawa et al., “Adiponectin and Metabolic Syndrome, Arterioscler Thromb Vasc Biol. 2004; 24:29-33). In healthy subjects, adiponectin carries out its roles for preventing development of vascular changes and the impairment of glucose and lipid metabolism, which may be induced by a variety of attacking factors, such as chemical subjects, mechanical stress, or nutritional loading. The above mentioned article by Matsuzawa et al., “Adiponectin and Metabolic Syndrome” suggests that adiponectin may play a key role in the prevention of metabolic syndrome. Hypoadiponectinemia observed in obesity, especially with visceral fat accumulation, is much more frequent than genetic hypoadiponectinemia. Hypoadiponectinemia together with the increase of PAI-1 induced by the accumulation of visceral obesity might be a major background of vascular changes as well as metabolic disorders.

In view of the above-discussed impact of adiponectin, specifically of hypoadiponectinemia, on one or more symptoms of the metabolic syndrome, it would be also desirable to find a method of influencing the level of expression or the concentration of adiponectin.

Metabolic syndrome can be prevented or treated by an appropriate, reduced calorie diet consisting of healthy foods (including proper amounts of dietary fiber) and by sufficient exercise (Deen et al., 2004, American Family Physician, V69/12, pp 2875-2882). However, many persons suffering from metabolic syndrome are unable to sufficiently change their dietary and exercise habits to prevent the syndrome or to emerge from the syndrome. Thus, there remains a need for a medicament, pharmaceutical composition, food, food ingredient or supplement, or nutraceutical ingredient or supplement to assist persons to prevent metabolic syndrome and to assist persons suffering from metabolic syndrome to emerge from this disease.

Several pharmaceutical compositions, nutraceutical ingredients and dietary supplements have been suggested for treating or preventing individual aspects of the metabolic syndrome.

WO 2004/022074 discloses the use of a composition comprising a non-glucose carbohydrate and soluble fiber or a mixture of pectin and soluble fiber for triggering the secrection of glucagen-like peptide 1. The publication lists a large variety of biological and medical indications like controlling metabolic syndrome, diabetes or obesity, or for the promotion of satiety, weight loss or maintenance of the desired body weight. Disclosed non-glucose carbohydrates are galactose, xylose, fructose or mannose. A large variety of soluble fibers is disclosed.

U.S. Pat. No. 5,576,306 discloses the use of water-soluble high-viscosity grades cellulose ether compositions for the reduction of serum lipid levels, particularly total serum cholesterol, serum triglycerides, and low-density lipoprotein (LDL) levels and/or attenuation of the postprandial rise of blood glucose levels in animals.

U.S. Pat. No. 5,585,366 discloses the use of water-soluble cellulose ethers, such as hydroxypropyl methyl cellulose, for reducing the cholesterol level in mammalian blood.

U.S. Pat. No. 6,899,892 discloses the use of water-soluble, non-nutritive, indigestible, non-starch, viscous polysaccharide, such as water-soluble cellulose ethers, for reducing the percentage of body fat and/or the leptin in the bloodstream of the mammal.

U.S. Pat. No. 5,721,221 discloses the use of hydroxypropyl methyl cellulose having a viscosity of 50 to 4,000 cps, measured as a 2 weight percent aqueous solution, for reducing total plasma cholesterol levels in a human.

Co-inventors of the present invention have published at the ACS (American Chemical Society) meeting, San Diego, Calif., Mar. 15, 2005 that hydroxypropylmethylcellulose (HPMC) may prevent insulin resistance in hamsters fed high saturated fat diets through regulating metabolic genes. Syrian hamsters fed a high fat diet similar in fat content to the American diet become insulin resistant (IR). Replacing cellulose in this high fat diet with hydroxypropylmethylcellulose significantly decreases the incidence of insulin resistance. HPMC significantly reduced the glucose infusion rate, fasting plasma insulin, plasma lipids, overall fat distribution in non-adipose tissues, and the cell size of adipose tissues.

The use of water-soluble METHOCEL dietary fiber for slowing fat absorption in a high-fat diet and its potential reduction in the development of insulin resistance, a precursor to Type II diabetes, has subsequently been advertised by The Dow Chemical Company based in the above-mentioned findings of the co-inventors of the present invention.

While the water-soluble cellulose ethers are very useful for the treatments disclosed above, they suffer from the problem of poor “mouth feel” because such water soluble cellulose ethers tend to form slimy viscous solutions with water. Moreover, it is sometimes not very easy to formulate and process water-soluble cellulose ethers into foods because of their viscosity, which is sometimes very high, especially in the presence of water.

Accordingly, it is one object of the present invention to find a compound or composition which is useful for preventing or treating at least one of the following abnormalities in an individual: abdominal obesity, atherogenic dyslipidemia, hypertension, insulin resistance with or without glucose intolerance, proinflammatory state and prothrombotic state.

It is a preferred object of the present invention to find a compound or composition which is useful for preventing or treating at least three of the above-mentioned abnormalities in an individual, specifically to find a compound or composition which is useful for preventing or treating metabolic syndrome or a symptom or condition associated with the metabolic syndrome in an individual.

It is another preferred object of the present invention to find a compound or composition which is useful to influence the level of expression or the concentration of C-reactive protein or of Plasminogen Activator Inhibitor-1 or both in a body tissue of an individual.

It is yet another preferred object of the present invention to find a compound or composition for one or more of the above-mentioned uses, which compound or composition does not tend to form a slimy viscous solution with water.

### SUMMARY OF THE INVENTION

It has surprisingly been found that water-insoluble cellulose derivatives, particularly ethyl cellulose, are useful for the prevention or treatment of one or more of the symptoms a) atherogenic dyslipidemia, b) insulin resistance, c) proinflammatory or inflammation state and d) prothrombotic state.

It has also surprisingly been found that water-insoluble cellulose derivatives are useful for preventing or treating metabolic syndrome or a symptom or condition associated with the metabolic syndrome, particularly a cardiovascular disease or type II diabetes in an individual.

More specifically, it has surprisingly been found that water-insoluble cellulose derivatives, particularly ethyl cellulose, are useful for influencing the level of expression of or the concentration of C-reactive protein (CRP), of Plasminogen Activator Inhibitor-1 (PAI-1), of hepatic lipase (HL) or of two or three thereof in a body tissue. A proinflammatory state, one of the symptoms of the metabolic syndrome, is recognized clinically by elevated concentration or level of expression of C-reactive protein (CRP).

While it is not fully clear yet whether CRP, PAI-1 and HL are only markers of one or more symptoms of metabolic syndrome or actually cause one or more of these symptoms, influencing their level in a body tissue, specifically reducing their level, is an important factor in the prevention or treatment of metabolic syndrome.

It has also surprisingly been found that water-insoluble cellulose derivatives, particularly ethyl cellulose, are useful for influencing the level of expression of or the concentration of adiponectin in a body tissue.

Based on evaluations of LDL cholesterol, VLDL cholesterol, Total Cholesterol and triglycerides in blood of individuals it has also been surprisingly been found that water-insoluble cellulose derivatives, particularly ethyl cellulose, are useful for the prevention or treatment of atherogenic dyslipidemia.

It is known that a fatty liver is closely associated with insulin resistance. Based on liver examinations of individuals it has also been surprisingly been found that water-insoluble cellulose derivatives, particularly ethyl cellulose, are useful for the prevention or treatment of insulin resistance.

Accordingly, one aspect of the present invention is a method of preventing or treating metabolic syndrome or a symptom or condition associated with the metabolic syndrome in an individual which comprises the step of administering to the individual an effective amount of a water-insoluble cellulose derivative.

Another aspect of the present invention is a method of preventing or treating one or more of the symptoms a) atherogenic dyslipidemia, b) insulin resistance, c) proinflammatory or inflammation state and d) prothrombotic state in an individual which comprises the step of administering to the individual an effective amount of a water-insoluble cellulose derivative.

Yet another aspect of the present invention is a method of influencing the level of expression or the concentration of C-reactive protein, of Plasminogen Activator Inhibitor-1, of hepatic lipase, or of adiponectin or of two or three thereof in a body tissue of an individual which comprises the step of administering to the individual an effective amount of a water-insoluble cellulose derivative.

Yet another aspect of the present invention is a method of preventing or treating a cardiovascular disease or Type II diabetes in an individual which comprises the step of administering to the individual an effective amount of a water-insoluble cellulose derivative.

Yet another aspect of the present invention is the use of a water-insoluble cellulose derivative for the manufacture of a medicament, pharmaceutical composition, food, food ingredient or supplement, or nutraceutical ingredient or supplement to prevent or treat metabolic syndrome or a symptom or condition associated with the metabolic syndrome in an individual.

Yet another aspect of the present invention is the use of a water-insoluble cellulose derivative for the manufacture of a medicament, pharmaceutical composition, food, food ingredient or supplement, or nutraceutical ingredient or supplement to prevent or treat one or more of the symptoms a) atherogenic dyslipidemia, b) insulin resistance, c) proinflammatory or inflammation state and d) prothrombotic state in an individual.

Yet another aspect of the present invention is the use of a water-insoluble cellulose derivative for the manufacture of a medicament, pharmaceutical composition, food, food ingredient or supplement, or nutraceutical ingredient or supplement to influence the level of expression or the concentration of C-reactive protein, of Plasminogen Activator Inhibitor-1, of hepatic lipase or of adiponectin or of two or three thereof in a body tissue of an individual.

Yet another aspect of the present invention is the use of a water-insoluble cellulose derivative for the manufacture of a medicament, pharmaceutical composition, food, or food ingredient or supplement, or nutraceutical ingredient or supplement to prevent or treat a cardiovascular disease or Type II diabetes in an individual.

Yet another aspect of the present invention is a medicament, pharmaceutical composition, food, food ingredient or supplement, or nutraceutical ingredient or supplement which comprises an effective amount of a water-insoluble cellulose derivative for preventing or treating metabolic syndrome or a symptom or condition associated with the metabolic syndrome.

Yet another aspect of the present invention is a medicament, pharmaceutical composition, food, food ingredient or supplement, or nutraceutical ingredient or supplement which comprises an effective amount of a water-insoluble cellulose derivative for preventing or treating one or more of the symptoms a) atherogenic dyslipidemia, b) insulin resistance, c) proinflammatory or inflammation state and d) prothrombotic state in an individual.

Yet another aspect of the present invention is a medicament, pharmaceutical composition, food, food ingredient or supplement, or nutraceutical ingredient or supplement which comprises an effective amount of a water-insoluble cellulose derivative for influencing the level of expression or the concentration of C-reactive protein, of Plasminogen Activator Inhibitor-1, of hepatic lipase or of adiponectin or of two or three thereof in a body tissue of an individual.

Yet another aspect of the present invention is a medicament, pharmaceutical composition, food, food ingredient or supplement, or nutraceutical ingredient or supplement which comprises an effective amount of a water-insoluble cellulose derivative for preventing or treating a cardiovascular disease or Type II diabetes in an individual.

Yet another aspect of the present invention is a medicament, pharmaceutical composition, food, food ingredient or supplement, or nutraceutical ingredient or supplement which comprises ethyl cellulose as an active principle.

Yet another aspect of the present invention is a water-insoluble cellulose derivative as a medicament for the prevention or treatment of metabolic syndrome or a symptom or condition associated with the metabolic syndrome.

Yet another aspect of the present invention is a water-insoluble cellulose derivative as a medicament for the prevention or treatment of one or more of the symptoms a) atherogenic dyslipidemia, b) insulin resistance, c) proinflammatory or inflammation state and d) prothrombotic state in an individual.

Yet another aspect of the present invention is a water-insoluble cellulose derivative as a medicament for influencing the level of expression or the concentration of C-reactive protein, of Plasminogen Activator Inhibitor-1, of hepatic lipase, or of adiponectin or of two or three thereof in a body tissue.

Yet another aspect of the present invention is a water-insoluble cellulose derivative as a medicament for the prevention or treatment of a cardiovascular disease or Type II diabetes in an individual.

### BRIEF DESCRIPTION OF THE DRAWINGS

FIG. 1 is a reproduction of a representative transmission electron micrograph at a magnification of 5,000× for a hamster liver after the hamster is fed a high fat diet containing microcrystalline cellulose; and

FIG. 2 is a reproduction of a representative transmission electron micrograph at a magnification of 5,000× for a hamster liver after the hamster is fed a high fat diet containing a water-insoluble cellulose derivative.

### DETAILED DESCRIPTION OF THE INVENTION

The term “metabolic syndrome” as used herein is characterized by at least three of the following abnormalities: abdominal obesity, atherogenic dyslipidemia, hypertension, insulin resistance with or without glucose intolerance, proinflammatory state and prothrombotic state.

The term “a symptom or condition associated with the metabolic syndrome” is defined herein as disclosed in the International Patent Application WO 2004/022074 comprises, but is not restricted to one or more symptoms or conditions selected from hyperglycemia, hyperinsulinaemia, hyperlipidaemia, impaired glucose metabolism, diabetic retinopathy, macular degeneration, cataracts, diabetic nephropathy, glomeruloscerosis, diabetic neuropathy, erectile dysfunction, premenstrual syndrome, vascular restenosis, and/or ulcerative colitis, angina pectoris, myocardial infection, stroke, skin and/or connective tissue disorders, foot ulcerations, metabolic acidosis, arthritis, osteoporosis and conditions of impaired glucose tolerance.

The term “a cardiovascular disease or Type II diabetes” includes the cardiovascular disease or Type II diabetes individually but also a cardiovascular disease and Type II diabetes in combination.

Abdominal obesity is generally characterized by excess body fat in the region of the abdomen.

The term hypertension is commonly known as high blood pressure.

Insulin resistance is generally characterized by an impaired ability of the body's insulin to regulate blood glucose metabolism.

Atherogenic dyslipidemia is generally characterized by increased low density lipoprotein [LDL] cholesterol and triglyceride levels and decreased high density lipoprotein [HDL] cholesterol level in blood.

As disclosed in U.S. Pat. No. 5,576,306, lipids are transported in the blood by the plasma lipoproteins. Lipoproteins (which account for 8% to 10% of the total serum protein) contain specific proteins (known as apolipoproteins), and varying amounts of cholesterol, triglycerides and phospholipids. The three major classes of lipoproteins found in the plasma in the fasting state are very low density lipoproteins (VLDL), low density lipoproteins (LDL) and high density lipoproteins (HDL). VLDLs contain over 50% triglyceride, about 20% cholesterol and about 10% protein. LDLs are much smaller particles and contain about 50% cholesterol, 20% protein and about 5% triglyceride. HDLs are the smallest of the lipoproteins and contain about 50% protein, 10% triglyceride and 20% cholesterol. In addition, chylomicrons, which are synthesized in the intestine in response to a fat-containing meal, appear transiently in the plasma and are cleared from the circulation within a few hours. They are not normally present in the fasting state, and contain about 90% by weight triglycerides, and 5% cholesterol. In the normal adult human, LDLs carry about 65% of the circulating cholesterol, HDLs carry about 25% and VLDLs carry about 10%.

The terms “a method of preventing or treating metabolic syndrome or a symptom or condition associated with the metabolic syndrome” and “a method of preventing or treating one or more of the symptoms a) atherogenic dyslipidemia, b) insulin resistance, c) proinflammatory or inflammation state and d) prothrombotic state” as used herein include any treatment that delays the development of an above-mentioned syndrome or symptom in time or in severity or that reduces the severity of a developing or developed syndrome or symptom.

The term “influencing the level of expression or the concentration of C-reactive protein, of Plasminogen Activator Inhibitor-1 or of hepatic lipase (HL) or of two or three thereof in a body tissue of an individual” means that the body tissue, such as blood, has a different, generally a lower, level of expression or concentration of CRP and/or PAI-1 and/or HL after the intake of a water-insoluble cellulose derivative by an individual, as compared to the level of expression or the concentration of CRP and/or PAI-1 and/or HL after the intake of a non-effective material such as unmodified cellulose itself.

The term “influencing the level of expression or the concentration of C-reactive protein, of Plasminogen Activator Inhibitor-1 or of hepatic lipase (HL) or of two or three thereof in a body tissue of an individual” means that the body tissue, such as blood, has a different, generally a lower, level of expression or concentration of CRP and/or PAI-1 and/or HL after the intake of a water-insoluble cellulose derivative by an individual, as compared to the level of expression or the concentration of CRP and/or PAI-1 and/or HL after the intake of a non-effective material such as unmodified cellulose itself.

The term “influencing the level of expression of CRP and/or PAI-1 and/or HL” is not limited to the direct regulation of the expression of CRP and/or PAI-1 and/or HL but also includes the indirect influence on CRP and/or PAI-1 and/or HL expression, for example by influencing the conditions or metabolites in a body tissue which lead to a different, preferably lower gene expression.

The term “influencing the level of expression or the concentration of adiponectin in a body tissue of an individual” means that the body tissue, such as blood, has a different, generally a higher, level of expression or the concentration of adiponectin after the intake of a water-insoluble cellulose derivative by an individual, as compared to the level of expression or the concentration of adiponectin after the intake of a non-effective material such as unmodified cellulose itself.

The term “influencing the level of expression of adiponectin” is not limited to the direct regulation of the expression of adiponectin also includes the indirect influence on adiponectin expression, for example by influencing the conditions or metabolites in a body tissue which lead to a different, preferably higher gene expression.

The present invention relates to the treatment of individuals, that means any animals including human beings. Preferred individuals are mammals. The term “mammal” refers to any animal classified as a mammal, including human beings, domestic and farm animals, such as cows, nonhuman primates, zoo animals, sports animals, such as horses, or pet animals, such as dogs and cats.

The cellulose derivatives which are useful in the present invention are water-insoluble. The term “cellulose derivative” does not include unmodified cellulose itself which also tends to be water-insoluble. Experiments conducted by the Applicants have shown that water-insoluble cellulose derivatives have a significantly different effect on the prevention or treatment of the metabolic syndrome or a symptom or condition associated with the metabolic syndrome than unmodified cellulose.

The term “water-insoluble” as used herein means that the cellulose derivative has a solubility in water of less than 2 grams, preferably less than 1 gram, in 100 grams of distilled water at 25° C. and 1 atmosphere.

Preferred cellulose derivatives for use in the present invention are water-insoluble cellulose ethers, particularly ethyl cellulose, propyl cellulose or butyl cellulose. Other useful water-insoluble cellulose derivatives are cellulose derivatives which have been chemically, preferably hydrophobically, modified to provide water insolubility. Chemical modification can be achieved with hydrophobic long chain branched or non-branched alkyl, arylalkyl or alkylaryl groups. “Long chain” typically means at least 5, more typically at least 10, particularly at least 12 carbon atoms. Others type of water-insoluble cellulose are crosslinked cellulose, when various crosslinking agents are used. Chemically modified, including the hydrophobically modified, water-insoluble cellulose derivatives are known in the art. They are useful provided that they have a solubility in water of less than 2 grams, preferably less than 1 gram, in 100 grams of distilled water at 25° C. and 1 atmosphere. The most preferred cellulose derivative is ethyl cellulose. The ethyl cellulose preferably has an ethoxyl substitution of from 40 to 55 percent, more preferably from 43 to 53 percent, most preferably from 44 to 51 percent. The percent ethoxyl substitution is based on the weight of the substituted product and determined according to a Zeisel gas chromatographic technique as described in ASTM D4794-94 (2003). The molecular weight of the ethyl cellulose is expressed as the viscosity of a 5 weight percent solution of the ethyl cellulose measured at 25° C. in a mixture of 80 volume percent toluene and 20 volume percent ethanol. The ethyl cellulose concentration is based on the total weight of toluene, ethanol and ethyl cellulose. The viscosity is measured using Ubbelohde tubes as outlined in ASTM D914-00 and as further described in ASTM D446-04, which is referenced in ASTM D914-00. The ethyl cellulose generally has a viscosity of up to 400 mPa·s, preferably up to 300 mPa·s, more preferably up to 100 mPa·s, measured as a 5 weight percent solution at 25° C. in a mixture of 80 volume percent toluene and 20 volume percent ethanol. The preferred ethyl celluloses are premium grades ETHOCEL ethyl cellulose which are commercially available from The Dow Chemical Company of Midland, Mich. Combinations of two or more water-insoluble cellulose derivatives are also useful.

Preferably the water-insoluble cellulose derivative has an average particle size of less than 0.1 millimeter, more preferably less than 0.05 millimeter, most preferably less than 0.02 millimeter. Preferably the water-insoluble cellulose derivative is exposed to an edible fat or oil before being administered to an individual so that the cellulose derivative imbibes the fat or oil. Advantageously the water-insoluble cellulose derivative is exposed to an excess of the fat or oil at about 40 to 60° C.

In the preferred embodiments of the present invention the water-insoluble cellulose derivatives, particularly ethyl cellulose, are useful for the prevention or treatment of at least two, more preferably at least three of the symptoms a) atherogenic dyslipidemia, b) insulin resistance, c) proinflammatory or inflammation state and d) prothrombotic state.

Furthermore, in the preferred embodiments of the present invention the water-insoluble cellulose derivatives, particularly ethyl cellulose, are useful for influencing the level of expression or the concentration of C-reactive protein (CRP) and of hepatic lipase (HL).

The water-insoluble cellulose derivative can be administered or consumed in or as a medicament, pharmaceutical composition, food, food ingredient or supplement, or nutraceutical ingredient or supplement. The medicament, pharmaceutical composition, food, food ingredient or supplement, or nutraceutical ingredient or supplement can be solid or liquid. The desired time period of administering the water-insoluble cellulose derivative can vary depending on the amount of water-insoluble cellulose derivative consumed, the general health of the individual, the level of activity of the individual and related factors. Since metabolic syndrome or a symptom or condition associated with metabolic syndrome is typically induced by an imbalanced nutrition with a high fat content, it may be advisable to administer or consume the water-insoluble cellulose derivative as long as nutrition with a high fat content is consumed. Generally administration of at least 1 to 12 weeks, preferably 3 to 8 weeks is recommended.

It is to be understood that the duration and daily dosages of administration as disclosed herein are general ranges and may vary depending on various factors, such as the specific cellulose derivative, the weight, age and health condition of the individual, and the like. It is advisable to follow the prescriptions or advices of medical doctors or nutrition specialists when consuming the water-insoluble cellulose derivatives.

According to the present invention the water-insoluble cellulose derivatives are preferably used for preparing food, a food ingredient or supplement, or a nutraceutical ingredient or supplement which comprises from 0.5 to 20 weight percent, more preferably from 2 to 15 weight percent, most preferably from 4 to 12 weight percentage of one or more water-insoluble cellulose derivatives. The given weight percentages relate to the total amount of the water-insoluble cellulose derivatives. The amount administered is preferably in the range of from 1 to 10 percent of the total daily weight of the diet of the individual on a dry weight basis. Preferably, the water-insoluble cellulose derivative is administered or consumed in sufficient amounts throughout the day, rather than in a single dose or amount. When the water-insoluble cellulose derivatives are administered or consumed in combination with water, the water-insoluble cellulose derivatives will generally not suffer from the “mouth feel” compliance issues, which are sometimes created by water-soluble cellulose derivatives due to their tendency to form slimy viscous solutions with water. Although the water-insoluble cellulose derivatives are preferably administered in combination with food or as foodstuff, alternatively they can be administered as an aqueous suspension or in powder form or as pharmaceutical or nutraceutical compositions. Pharmaceutical or nutraceutical compositions containing water-insoluble cellulose derivatives can be administered with an acceptable carrier in a pharmaceutical or nutraceutical unit dosage form. Pharmaceutically acceptable carriers include tableting excipients, gelatin capsules, or carriers such as a polyethylene glycol or a natural gel. Pharmaceutical or nutraceutical unit dosage forms include tablets, capsules, gelatin capsules, pre-measured powders and pre-measured solutions. Hence, the water-insoluble cellulose derivatives preferably are formulated as tablets, granules, capsules and suspensions.

Regardless whether the water-insoluble cellulose derivative is administered as an aqueous suspension or in powder form, as a pharmaceutical or nutraceutical composition or is combined with other food ingredients, the amount of administered water-insoluble cellulose derivative is generally in the range of from 10 to 300 milligrams of water-insoluble cellulose derivative per pound of mammal body weight per day. About 2 g to about 30 g, preferably about 3 g to about 15 g of water-insoluble cellulose derivative are ingested daily by a large mammal such as a human.

While the method of administration or consumption may vary, the water-insoluble cellulose derivatives are preferably ingested by a human as a food ingredient of his or her daily diet. The water-insoluble cellulose derivatives can be combined with a liquid vehicle, such as water, milk, vegetable oil, juice and the like, or with an ingestible solid or semi-solid foodstuff, such as “veggie” burgers, spreads or bakery products.

A number of foodstuffs are generally compatible with water-insoluble cellulose derivatives. For example, a water-insoluble cellulose derivative may be mixed into foods such as milk shakes, milk shake mixes, breakfast drinks, juices, flavored drinks, flavored drink mixes, yoghurts, puddings, ice creams, ice milks, frostings, frozen yoghurts, cheesecake fillings, candy bars, including “health bars” such as granola and fruit bars, gums, hard candy, mayonnaise, pastry fillings such as fruit fillings or cream fillings, cereals, breads, stuffing, dressings and instant potato mixes. An effective amount of water-insoluble cellulose derivatives can also be used as a fat-substitute or fat-supplement in salad dressings, frostings, margarines, soups, sauces, gravies, mustards and other spreads. Therefore, “food ingredients,” as the term is used herein, includes those ingredients commonly employed in recipes for the above foodstuffs, including, for example, flour, oatmeal, fruits, milk, eggs, starch, soy protein, sugar, sugar syrups, vegetable oils, butter or emulsifying agents such as lecithin. Colorings and flavorings may be added as may be appropriate to add to the attractiveness of the foodstuff.

The water-insoluble cellulose derivative can also be administered to domestic and farm animals, such as cows, nonhuman primates, zoo animals, sports animals, such as horses, or pet animals, such as dogs and cats, in a known manner in or as a medicament, pharmaceutical composition, food, food ingredient or supplement, or nutraceutical ingredient or supplement. A preferred way of administration is the incorporation of a water-insoluble cellulose derivative in the pet feed or other animal feed.

The water-insoluble cellulose derivative is optionally used in combination with water-soluble or water-insoluble naturally occurring polymers or derivatives thereof, such as gum arabic, xanthan gum or derivatives thereof, gum karaya, gum tragacanth, gum ghatti, guar gum or derivatives thereof, exudate gums, seaweed gums, seed gums, microbial gums, carrageenan, dextran, gelatin, alginates, pectins, starches or derivatives thereof, chitosans or other polysaccharides, preferably beta-glucans, galactomannans, hemicelluloses, psyllium, guar, xanthan, microcrystalline cellulose, amorphous cellulose or chitosan.

In some embodiments of the present invention it is particularly beneficial to use or administer a water-insoluble cellulose derivative in combination with a water-soluble cellulose derivative. Useful amounts of combinations of one or more water-insoluble cellulose derivatives and one or more water-soluble cellulose derivatives and useful ways for administration, consumption or inclusion of such combinations in a medicament, pharmaceutical composition, food, food ingredient or supplement, or nutraceutical ingredient or supplement are generally the same as those described above for the water-insoluble cellulose derivatives alone.

The water-soluble cellulose derivatives have a solubility in water of at least 2 grams, preferably at least 3 grams, more preferably at least 5 grams in 100 grams of distilled water at 25° C. and 1 atmosphere. Preferred water-soluble cellulose derivatives are water-soluble cellulose esters and cellulose ethers. Preferred cellulose ethers are water-soluble carboxy-C₁-C₃-alkyl celluloses, such as carboxymethyl celluloses; water-soluble carboxy-C₁-C₃-alkyl hydroxy-C₁-C₃-alkyl celluloses, such as carboxymethyl hydroxyethyl celluloses; water-soluble C₁-C₃-alkyl celluloses, such as methylcelluloses; water-soluble C₁-C₃-alkyl hydroxy-C₁-C₃-alkyl celluloses, such as hydroxyethyl methylcelluloses, hydroxypropyl methylcelluloses or ethyl hydroxyethyl celluloses; water-soluble hydroxy-C₁₋₃-alkyl celluloses, such as hydroxyethyl celluloses or hydroxypropyl celluloses; water-soluble mixed hydroxy-C₁-C₃-alkyl celluloses, such as hydroxyethyl hydroxypropyl celluloses, water-soluble mixed C₁-C₃-alkyl celluloses, such as methyl ethyl celluloses, or water-soluble alkoxy hydroxyethyl hydroxypropyl celluloses, the alkoxy group being straight-chain or branched and containing 2 to 8 carbon atoms. The more preferred cellulose ethers are methylcellulose, methyl ethyl cellulose, hydroxyethyl cellulose, hydroxypropyl cellulose, hydroxyethyl methylcellulose, hydroxypropyl methylcellulose, and carboxymethyl cellulose, which are classified as water-soluble cellulose ethers by the skilled artisans. The most preferred water-soluble cellulose ethers are methylcelluloses with a methyl molar substitution DS_{methoxyl} of from 0.5 to 3.0, preferably from 1 to 2.5, and hydroxypropyl methylcelluloses with a DS_{methoxyl} of from 0.9 to 2.2, preferably from 1.1 to 2.0, and a MS_{hydroxypropoxyl} of from 0.02 to 2.0, preferably from 0.1 to 1.2. The methoxyl content of methyl cellulose can be determined according to ASTM method D 1347-72 (reapproved 1995). The methoxyl and hydroxypropoxyl content of hydroxypropyl methylcellulose can be determined by ASTM method D-2363-79 (reapproved 1989). Methyl celluloses and hydroxypropyl methylcelluloses, such as K100M, K4M, K1M, F220M, F4M and J4M hydroxypropyl methylcellulose are commercially available from The Dow Chemical Company). The water-soluble cellulose derivative generally has a viscosity of from 5 to 2,000,000 cps (=mPa·s), preferably from 50 cps to 200,000 cps, more preferably fromt 75 to 100,000 cps, in particular from 1,000 to 50,000 cps, measured as a two weight percent aqueous solution at 20 degrees Celsius. The viscosity can be measured in a rotational viscometer.

The present invention is further illustrated by the following examples which are not to be construed to limit the scope of the invention. Unless otherwise mentioned, all parts and percentages are by weight.

### EXAMPLES

Very low density lipoprotein (VLDL), low density lipoprotein (LDL) and high density lipoprotein (HDL) cholesterol levels in the blood were determined according to size-exclusion chromatography (SEC) method, which allowed separation and simultaneous determination of cholesterol lipoproteins, based upon their particle size. Agilent 1100 chromatograph was employed with a post-column derivatization reactor, consisting of a mixing coil in a temperature-controlled water jacket and a Hewlett-Packard HPLC pump 79851-A, was used to deliver cholesterol reagent at a flow rate of 0.2 ml/min. Cholesterol lipoprotein standards (bovine) were used to calibrate the UV detector. Calibration was performed using standard peak areas. Typically, blood was collected via cardiac puncture, into 5 ml syringes, rinsed with potassium EDTA solution, through a 21-gram needle. The blood was transferred to 5 ml polypropylene tubes (containing potassium EDTA solution to prevent coagulation) and placed on a rocker for a few minutes, then stored on ice until centrifugation. Centrifugation was then performed at 1,500 rpm for 30 min at 4° C., using a commercial clinical centrifuge. The aliquots of plasma (supernatant) were transferred to the Eppendorf tubes and 15 μl of plasma was injected via the Agilent 1100 autosampler onto a Superose 6HR HPLC column. The lipoproteins were eluted with a buffer containing 0.15 M NaCl (pH 7.0, 0.02% sodium azide) at a flow rate of 0.5 ml/min. Identical instrumental setup and SEC determination method were applied for analysis of triglycerides (TG) in blood, but with a different post-column derivatization reagent. The total cholesterol (TC) level was obtained by summarizing the VLDL, LDL and HDL levels. The sum of VLDL and LDL levels (VLDL+LDL) was also utilized to illustrate the level of overall “bad” cholesterol in blood.

The weight percent fat content of the livers of the hamsters was also determined. Freshly removed livers were frozen immediately in liquid nitrogen. A small section of the frozen livers were freeze-dried for fat analysis. The freeze-dried livers were mechanically crushed into fine powder, while stored in a small Ziploc™ bags. Exactly 200 mg of the lyophilized liver powder was then extracted using Hexane/Isopropanol blend (3:2). The Dionex ASE 200 extractor was used. The extract was subject to solvent evaporation and a subsequent gravimetric analysis for the fat content determination.

The powdered ethyl cellulose used in the Examples is commercially available from The Dow Chemical Company under the trademark ETHOCEL Standard 10 Premium and ETHOCEL Standard 10 Premium FP grade. Ethocel Standard 10 Premium has considerably larger particles than Ethocel Standard 10 Premium FP, hence the first is herein referred to as “coarse” particles and the second is herein referred to as “fine” particles. It has an ethoxyl content of 48.0-49.5 percent and a viscosity of about 10 mPa·s, measured as a 5 weight percent solution at 25° C. in a mixture of 80 volume percent toluene and 20 volume percent ethanol using a Brookfield viscometer.

### Procedure of Examples 1 and 2

An animal study was conducted with male golden Syrian hamsters with a starting body weight of 70-90 grams (Sasco strain, Charles River, Wilmington, Mass.). The animal study was approved by the Animal Care and Use Committee, Western Regional Research Center, USDA, Albany, Calif. The male Syrian golden hamsters were divided into two groups: one of the groups was called “Treatment Group” and was fed a high-fat treatment diet and water ad libitum, while the second of the groups was called “Control Group” and was fed high-fat control diet and water ad libitum. Each group counted 10 hamsters. Both groups were fed for a period of three consecutive weeks.

A water-insoluble cellulose ether was present at 5 weight percent level in the treatment diet. It was first suspended in liquefied fat components of the diet, before mixing with the powdered components of the diet. 1000 g of the complete high-fat treatment diet contained 80 g of butter fat, 100 g of corn oil, 20 g of fish oil and 1 g of cholesterol, 200 g of casein, 498 g of corn starch, 3 g of DL methionine, 3 g of choline bitartrate, 35 g of a mineral mixture, 10 g of a vitamin mixture and 50 g of ETHOCEL Standard Premium 10 “coarse” grade ethyl cellulose.

The control diet had exactly the same composition as the treatment diet, with the only exception that the water-insoluble cellulose derivative was replaced by the same amount of microcrystalline cellulose (MCC), mixed into powdered components of the diet during the control diet preparation.

### Example 1

After the hamsters had been fed the diets for three consecutive weeks, the blood samples were taken from the hamsters to obtain blood plasma. Blood plasma was analyzed for cholesterol lipoprotein and triglycerides levels. The results are listed in Table 1 below.

TABLE 1

| Example 1 | Treatment Group | Control Group |
|---|---|---|
| LDL | 125.8 mg/dL ± 13.3 mg/dL | 176.7 mg/dL ± 14.2 mg/dL |
| cholesterol | | |
| VLDL | 29.7 mg/dL ± 2.2 mg/dL | 54.3 mg/dL ± 5.6 mg/dL |
| cholesterol | | |
| Total | 254.2 mg/dL ± 13.3 mg/dL | 339.8 mg/dL ± 14.5 mg/dL |
| Cholesterol | | |
| (TC) | | |
| Triglyc- | 78 mg/dL ± 4 mg/dL | 108 ± 14 mg/dL |
| erides (TG) | | |

The results in Example 1 are an indication that water-insoluble cellulose derivatives such as ethyl cellulose are useful for preventing or treating atherogenic dyslipidemia in an individual.

### Example 2

After the hamsters had been fed the diets for three consecutive weeks, the livers were taken out and the weight percent fat content of the livers of the sacrificed hamsters was determined gravimetrically, as described above. The results are listed in Table 2 below.

TABLE 2

| Example 2 | Treatment Group | Control Group |
|---|---|---|
| Fat Content of Livers | 0.167 g ± 0.008 g | 0.211 g ± 0.004 g |

### Example 3

The procedure for Example 3 was very similar to the procedure for the Examples 1 and 2. Male Syrian golden hamsters of the same strain and with the same range of starting body weight as in Examples 1 and 2 were also divided into the treatment group and the control group, 5 hamsters per each group. They were fed the diets that were the same as diets listed in the Examples 1 and 2, except that the treatment diet contained 3 weight percent of ETHOCEL Standard Premium 10 “coarse” grade ethyl cellulose and that this ethyl cellulose was mixed with powdered components of the diet with extra addition of ca. 300 g of water, then it was mixed with liquefied fat fraction of the diet. The control diet contained 3 weight percent of microcrystalline cellulose instead of the water-insoluble cellulose derivative.

After the hamsters had been fed the diets for three consecutive weeks, the livers were removed and the weight percent fat content of the livers of the sacrificed hamsters was determined gravimetrically, as described above. The fat content of the livers was calculated as a weight % of the livers of the hamsters. The results are listed in Table 3 below.

TABLE 3

| Example 3 | Treatment Group | Control Group |
|---|---|---|
| Fat Content of Livers, | 14.1% ± 0.8% | 18.1% ± 0.8% |
| as weight % of livers | | |

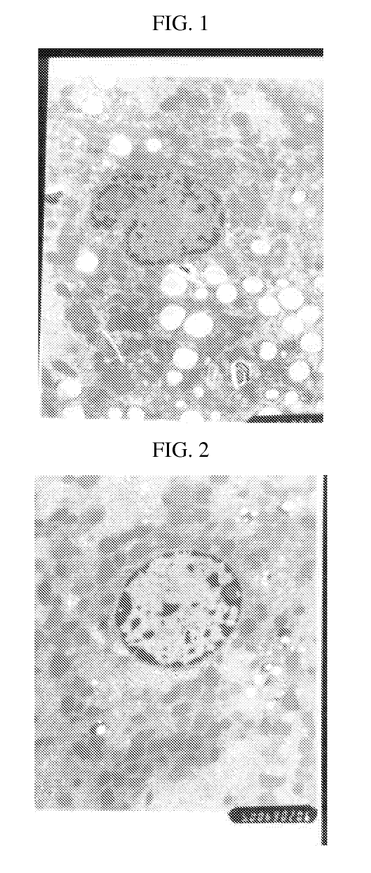

FIG. 1 shows a representative transmission electron micrograph at a magnification of 5,000× for a liver of a hamster fed control diet. FIG. 2 shows a representative transmission electron micrograph at a magnification of 5,000× for a liver of a hamster fed treatment diet. Referring now to FIG. 1 it will be noted that the liver cell nucleus is not well formed, that the membrane of the cell nucleus is abnormal and that the liver tissue contains numerous fat globules. Referring now to FIG. 2 in comparison to FIG. 1, it will be noted that in FIG. 2 the liver cell nucleus is well formed, that the membrane of the cell nucleus has a normal appearance and that the liver tissue shown in FIG. 2 contains fewer and smaller fat globules thereby indicating a favorable outcome for the diet containing the water-insoluble cellulose derivate.

It is known that a fatty liver is closely associated with insulin resistance, and in general the metabolic syndrome, see for example the following publications: 1) Knobler, H, Schattner A, Zhornicki T, Malnick S D H, Keter D, Sokolovskaya N, Lurie Y, and Bass D D, Fatty liver—an additional and treatable feature of the insulin resistance syndrome, Q J Med 92: 73-79, 1999; 2) Nguyen-Duy T B, Nichaman M Z, Church T S, Blair S N, and Ross R, Visceral fat and liver fat are independent predictors of metabolic risk factors in men, Am J Physiol Endocrinol Metab284: E1065-E1071, 2003; 3) Marchesini G, Brizi M, Bianchi G, Tomassetti S, Bugianesi E, Lenzi M, McCullough A J, Natale S, Forlani G, and Melchionda N, Nonalcoholic Fatty Liver Diease—A Feature of the Metabolic Syndrome, Diabetes 50: 1844-1850, 2001; 4) Garg A and Misra A, Hepatic Steatosis, Insulin Resistance, and Adipose Tissue Disorders, J Clin Endocrinol Metab 87(7): 3019-3022, 2002.

The results in Examples 2 and 3 are an indication that water-insoluble cellulose derivatives such as ethyl cellulose are useful for preventing or treating insulin resistance.

### Example 4

Male Syrian golden hamsters of the same strain and with the same range of starting body weight as in Examples 1 and 2 were divided into three groups. One of the groups was called “treatment group D” and was fed a high-fat treatment diet of one type and water ad libitum, the second group groups was called “treatment group F” and was fed a high-fat treatment diet of another type and water ad libitum, while the third of the groups was called “control group” and was fed high-fat control diet and water ad libitum. The treatment group D and treatment group F counted 10 hamsters each, while the control group counted 12 hamsters. The three groups were fed for a period of three consecutive weeks.

A water-insoluble cellulose ether was present at 5 weight percent level in the treatment diets D and F. In case of treatment diet D, water-insoluble cellulose ether was first mixed into the powdered components of the diet before blending it with the liquefied fat components of the diet. In case of treatment diet F, water-insoluble cellulose ether was first suspended in liquefied fat fraction of the diet, before mixing with the powdered fractions of the diet. For both treatment diets, D and F, a 1000 g of either of the complete high-fat treatment diets contained 80 g of butter fat, 100 g of corn oil, 20 g of fish oil and 1 g of cholesterol, 200 g of casein, 498 g of corn starch, 3 g of DL methionine, 3 g of choline bitartrate, 35 g of a mineral mixture, 10 g of a vitamin mixture and 50 g of ETHOCEL Standard Premium 10 FP “fine” grade ethyl cellulose.

The control diet had exactly same composition as treatment diet, with the only exception that the water-insoluble cellulose derivative was replaced by same amount of microcrystalline cellulose (MCC), mixed into powdered components of the diet during the control diet preparation.

After the hamsters had been fed the diets for three consecutive weeks, the blood samples were taken from the hamsters to obtain blood plasma. Blood plasma was analyzed for cholesterol lipoprotein and triglycerides levels. The LDL, VLDL and TC (Total Cholesterol) levels were measured, and determined as indicated above in the Example 1. The results are listed in Table 4 below.

TABLE 4

| | Treatment | | Treatment | | Control | |
|---|---|---|---|---|---|---|
| Example 4 | group D | | group F | | group | |
| VLDL | 13.7 | (±2.0) | 10.4 | (±1.9) | 36.6 | (±4.2) |
| LDL | 88.3 | (±8.2) | 58.4 | (±4.7) | 175.0 | (±11.0) |
| VLDL + LDL | 102.0 | (±8.9) | 68.7 | (±5.9) | 211.6 | (±11.5) |
| TC | 222.4 | (±11.5) | 184.4 | (±10.0) | 331.8 | (±11.6) |

The results of Example 4 confirm the results of Example 1. For instance, the Total Cholesterol level in the blood plasma of an individual is significantly lower after the individual has consumed a high-fat diet comprising ethyl cellulose than after the individual has consumed a corresponding high-fat diet comprising microcrystalline cellulose instead of ethyl cellulose.

### Example 5

Male Syrian golden hamsters of the same strain and with the same range of starting body weight as in Examples 1 and 2 were divided into two groups. One of the groups was called “treatment group” and was fed a high-fat treatment diet and water ad libitum, while the other group was called “control group” and was fed high-fat control diet and water ad libitum. Both groups counted 10 hamsters each. These groups were fed for a period of eight consecutive weeks.

A water-insoluble cellulose ether was present at 5 weight percent level in the treatment diet. In case this treatment diet, water-insoluble cellulose ether was first suspended in liquefied fat fraction of the diet, before mixing with the powdered fractions of the diet. For this treatment diet, a 1000 g of either of the complete high-fat treatment diets contained 150 g of butter fat, 50 g of corn oil, 200 g of casein, 499 g of corn starch, 3 g of DL methionine, 3 g of choline bitartrate, 35 g of a mineral mixture, 10 g of a vitamin mixture and 50 g of ETHOCEL Standard Premium 10 FP “fine” grade ethyl cellulose.

The control diet had exactly same composition as the treatment diet, with the only exception that the water-insoluble cellulose derivative was replaced by a same amount of microcrystalline cellulose (MCC), mixed into powdered components of the diet during the control diet preparation.

After the hamsters had been fed the diets for eight consecutive weeks, the livers were taken out from animals of the treatment group and animals of the control group on a random basis. The hamsters of the treatment group are designated in Table 5 below as HF-EC-1, HF-EC-2, HF-EC-3, HF-EC-4, HF-EC-5, HF-EC-6 and HF-EC-7. The hamsters of the control group are designated in Table 5 below as HF-Control-1 and HF-Control-2, HF-Control-3 and HF-Control-4.

Messenger ribonucleic acid (mRNA) was extracted from these livers of these hamsters. Total mRNA was extracted, purified, and reverse transcribed according to Bartley and Ishida (2002). The teaching of Bartley, G. E. and Ishida, B. K. (2002) Digital Fruit Ripening Data Mining in the TIGR Tomato Gene Index. Plant Mol. Biol. Rep. 20: 115-130, is included herein by reference.

cDNAs resulting from reverse transcription of the above total mRNAs were diluted 10 fold and 1 microliter aliquots were used in real-time PCR reactions with specific primers for the genes having a length of 20-24 bases as described further below and SYBR Green Supermix (BIO-RAD) according to the manufacturer's protocols with the following changes: 1. Reactions were performed in 25-microliter total volume in triplicate reactions 2. An MX3000P (Stratagene) instrument was used to perform the PCR. PCR conditions were 5 min at 95° C. followed by 40 cycles of incubation at 94° C.×15 s, 55 to 60° C.×1 min and 72° C.×30 s. The following primers were used:

| | |
|---|---|
| CRP: | CGTGTTGTCATTATGTAGGTCTTA (forward), |
| | GTAGCTTTATTGACTCATGGACC (reverse); |
| PAI-1: | TTCACAAGTCTTTCCGACCAA (forward), |
| | GGGGGCCATGCGGGCTGAGA (reverse); |
| HL: | AAGAGAATTCCCATCACCCTG (forward), |
| | CTGTTTTCCCACTTGAACTTGA (reverse); |
| Actin: | ACGTCGACATCCGCAAAGACCTC (forward), |
| | GATCTCCTTCTGCATCCGGTCA (reverse). |

Primer efficiencies were determined using dilution curves of cDNA. Relative quantitation was performed by normalization to the actin transcript as in Livak, K. J. and Schmittgen, T. D. (2001). The teaching of Livak, K. J. and Schmittgen, T. D. (2001), Analysis of relative gene expression data using real-time quantitative PCR and the 2^{-ΔΔC}T Method. Methods. 25: 402-408, is incorporated herein by reference. Negative controls to determine the extent of DNA contamination were carried out with identical concentrations of total mRNAs (samples after purification) without reverse transcription. A negative control was run for some of the primer sets. In each case the no-reverse transcription control signal was achieved after 5 or more cycles than the samples that were transcribed.

The C-reactive protein (CRP), Plasminogen Activator Inhibitor-1 (PAI-1) and hepatic lipase (HL) gene expression of the hamster HF-EC-1 was compared with the CRP, PAI and HL gene expression of the hamsters HF-Control-1 and HF-Control-2. The ratios for the gene expressions HF-EC-1/HF-Control-1 and HF-EC-1/HF-Control-2 are listed in Table 5 below. The ratios for the CRP, PAI and HL gene expression of the other pairs of hamsters were determined as listed in Table 5 below. It is understood that the numbers expressed in the Table 5 are relative to control, i.e. if the number is lower than 1 then the expression of a particular gene is lower in the hamsters from the treatment group than in the hamsters from the control group, and vice versa.

The results are listed in Table 5 below. The values in Table 5 for each animal pair and each gene are an average of triplicate measurements. The mean and standard error of the mean (SEM) values are given.

TABLE 5

| Animal pairs, ratio | | | |
|---|---|---|---|
| of gene expression | | | |
| after 8 weeks feeding | CRP | PAI-1 | HL |
| HF-EC-1/HF-Control-1 | 0.88 ± 0.23 | 0.68 ± 0.12 | 0.64 ± 0.12 |
| HF-EC-1/HF-Control-2 | 0.66 ± 0.17 | 0.64 ± 0.12 | 0.66 ± 0.04 |
| HF-EC-2/HF-Control-1 | 0.99 ± 0.28 | 0.93 ± 0.25 | 0.58 ± 0.10 |
| HF-EC-2/HF-Control-2 | 0.76 ± 0.27 | 0.86 ± 0.07 | 0.61 ± 0.05 |
| HF-EC-3/HF-Control-3 | 0.46 ± 0.02 | 1.3 ± 0.34 | 0.61 ± 0.56 |
| HF-EC-3/HF-Control-4 | 0.89 ± 0.2 | 0.79 ± 0.04 | 0.84 ± 0.25 |
| HF-EC-4/HF-Control-3 | 0.51 ± 0.17 | 2.0 ± 0.9* | 1.4 ± 0.76* |
| HF-EC-4/HF-Control-4 | 0.96 ± 0.22 | 1.2 ± 0.32 | 1.0 ± 0.12 |
| HF-EC-5/HF-Control-3 | 0.84 ± 0.09 | 0.7 ± 0.07 | Not measured |
| HF-EC-5/HF-Control-4 | 1.49 ± 0.1 | 0.55 ± 0.14 | Not measured |
| HF-EC-6/HF-Control-3 | 0.71 ± 0.13 | 0.72 ± 0.17 | Not measured |
| HF-EC-6/HF-Control-4 | 1.26 ± 0.26 | 0.55 ± 0.06 | Not measured |
| HF-EC-7/HF-Control-3 | 0.52 ± 0.05 | 1.2 ± 0.39 | Not measured |
| HF-EC-7/HF-Control-4 | 0.92 ± 0.1 | 0.91 ± 0.23 | Not measured |
| Mean | 0.85 | 0.85 | 0.71 |
| SEM (Standard Error | 0.08 | 0.07 | 0.14 |
| of Mean) | | | |

*Eliminated for calculating Mean and SEM based on “Standard Practice for Dealing With Outlying Observations” ASTM E 178-80. A statistical outlier analysis was done using the Grubb's analysis [Grubbs, Frank (February 1969), Procedures for Detecting Outlying Observations in Samples, Technometrics, Vol. 11, No. 1, pp. 1-21 and http://www.itl.nist.gov/div898/handbook/eda/section3/eda35h.htm].

While the data show some variation within the same group of animals, this is to be expected since the results are obtained on biological, living systems. Nevertheless, the data show a clear trend. The CRP, PAI-1 and HL gene expressions are generally lower in the animals of the Treatment Group that were fed a diet containing ethyl cellulose than in the animals of the Control Group that were fed a diet comprising microcrystalline cellulose instead of a water-insoluble cellulose derivative.

To reduce the CRP and/or PAI-1 and/or HL gene expression is an important factor in the prevention or treatment of metabolic syndrome.

### Example 6

An animal study was conducted with male golden Syrian hamsters with a starting body weight of 50-60 grams (LVG strain, Charles River, Wilmington, Mass.) in each of the diets specified below. The animal study was approved by the Animal Care and Use Committee, Western Regional Research Center, USDA, Albany, Calif.

The ethyl cellulose used in Example 6 was ETHOCEL Standard Premium 10 “fine” grade ethyl cellulose. It is commercially available from The Dow Chemical Company and has an ethoxyl content of 48.0-49.5 percent and a viscosity of about 10 mPa's, measured as a 5 weight percent solution at 25° C. in a mixture of 80 volume percent toluene and 20 volume percent ethanol using a Brookfield viscometer.

The male Syrian golden hamsters were divided into three groups. Two groups were called “treatment group” and were fed diets containing “EC dry” and “EC fat”. One group was called “control group” and was fed a diet consisting of microcrystalline cellulose (MCC). Each group consisted of approximately 10 hamsters each. These groups were fed for a period of three consecutive weeks.

### Treatment Group 1: EC Dry

This treatment group was fed a dry EC treatment diet as in Examples 1 and 2, except that int contained 50 g of ETHOCEL Standard Premium 10 FP “fine” grade ethyl cellulose.

### Treatment Group 2: EC Fat

The EC fat diet for Treatment Group 2 was the same as the diet for Treatment Group 1, except that the 50 g of ETHOCEL Standard Premium 10 FP “fine” grade ethyl cellulose was dispersed in the diet fat portion at 50° C. during the diet's preparation.

### Control Group: MCC

The control diet had exactly the same composition as treatment diet, with the only exception that the ethyl cellulose derivative was replaced by same amount of microcrystalline cellulose (MCC), mixed into powdered components of diet during the control diet preparation.

After the hamsters had been fed the diets for three consecutive weeks, plasma was obtained and the livers were taken out from both the treatment groups and control group. The sacrificed hamsters of the treatment group are designated in Table 6 below as “EC dry” and “EC fat”. The sacrificed hamsters of the control group are designated in Table 6 below as MCC.

### Quantitative RT-PCR Analysis PAI-1 in Hamster Livers

The gene expression for Plasminogen Activator Inhibitor-1 (PAI-1), was determined by mRNA extraction and analysis as described in Example 5. Total mRNA was extracted, purified, and reverse transcribed according to Bartley and Ishida (2002), as described in Example 5.

The PAI-1 gene expression of the hamster EC dry and EC fat were compared with PAI-1 gene expression of the hamster control MCC. The ratios for the gene expression are listed in Table 6 below. The mean and standard error of the mean (SEM) values are given. It is understood that the numbers expressed in the Table 6 are relative to control, i.e. if the number is lower than 1 then the expression of a particular gene is lower in hamsters from the treatment group than in the hamsters from the control group, and vice versa.

TABLE 6

| Ratio of Gene Expression | PAI-1 Mean (SEM) |
|---|---|
| EC dry/control MCC | 0.67 (0.14) |
| EC fat/control MCC | 0.63 (0.07) |

Table 6 illustrates that the administration of water-insoluble cellulose derivate, such as ethyl cellulose, has a significant effect on PAI-1 gene expression. Even though the diet was only three weeks the hamsters feed with the ethyl cellulose diet instead of microcrystalline cellulose had a significantly lower PAI-1 gene expression. The reduced PAI-1 gene expression is clear indication for the usefulness of water-insoluble cellulose derivate, such as ethyl cellulose, for prevention or treatment of metabolic syndrome.

### Analysis of Adiponectin in Hamster Plasma

Hamster EDTA plasma samples were assayed for adiponectin based on a double-antibody sandwich enzyme immunoassay technique.

Plasma samples were diluted prior to the start of the assay with reagent buffers from the Adiponectin ELISA Kit, B-Bridge International, Inc. (Mountain View, Calif.). After reconstituting all reagents, 100 μL of serially diluted adiponectin standards and diluted plasma sample were added to the appropriate number of antibody-coated wells. Adiponectin in the sample binds to the primary anti-adiponectin polycolonal antibody immobilized in the well (1^{st} reaction). The plates were incubated at 22-28° C. for 60 minutes. Following incubation each well was washed three times with the wash buffer. After washing, 100 μL of biotinylated secondary anti-adiponectin polyclonal antibody was added to each well and allowed to incubate at 22-28° C. for 60 minutes (2^{nd} reaction). The biotinylated secondary rabbit anti-adiponectin polyclonal antibody binds to the adiponectin trapped in the well in the 1^{st} Reaction. Following incubation each well was washed three times with the wash buffer. After washing, a conjugate of horseradish peroxidase (HRP) and streptavidin was added to each well and allowed to incubate at 22-28° C. for 60 minutes (Reaction 3). The HRP-conjugated streptavidin recognizes and binds to the biotinylated rabbit anti-adiponectin antibody trapped in the well in the 2^{nd} Reaction. After washing, the colorimetric substrate for the enzyme is added to all wells and incubated. The color development is terminated by the addition of a stop solution. The absorbance of each well was measured at 450 nm with a Synergy™ HT Multi-Detection Microplate Reader.

### Analysis of PAI-1 in Hamster Plasma

Hamster EDTA plasma samples were assayed for PAI-1 activity based on the inhibition of the plasminogen activator (urokinase (uPA) or tissue plasminogen activator (tPA)) activity of the synthetic chromogenic substrate method.

Plasma samples were assayed directly using the colorimetric assay of PAI-1 based on the procedures provided with the assay kit, STACHROM PAI, Diagnostica Stago (Parsippany, N.J.). A protocol for microplate format was used. After reconstituting all reagents, 25 μL of plasma or PAI calibrator and 100 μL at of Reagent 1 (uPA) were added to the designated wells. The plate was incubated in the pre-warmed plate reader at 37° C. for 4 minutes. This step initiated the binding between PAI-1 and uPA. For measuring the residual uPA activity after PAI-1 inhibition, 100 μL at of Reagent 2 (plasminogen) was added to each well and the reaction mixture was incubated at 37° C. for 4 minutes. Plasmin was generated as a result of the reaction, and the amidolytic activity of plasmin was determined by the reaction kinetics upon addition of 100 μL at of prewarmed substrate (Reagent 3) at 37° C. The absorbance at 405 nm was measured at 15 seconds and 45 seconds after the addition substrate. Because the assay was performed in the kinetic mode, the reagents should be added quickly and the precise time of each reagent addition should be noted. The PAI-1 level was determined based on the standard curve generated by plotting Δabsorbance value of the two time point versus the calibrator activity level provided with specific lot.

After the hamster plasma was obtained from the different diets the plasma was analyzed for PAI-1 and adiponectin. The PAI-1 and adiponectin protein levels were measured and determined. The results are listed in Table 7.

TABLE 7

| | | Ratio | | Ratio |
|---|---|---|---|---|
| | | EC dry/ | | EC fat/ |
| Diet | [PAI] | control MCC | [Adiponectin] | control MCC |
| Ec dry | 3.4 ± 1.5 | 0.76 | 11.0 ± 2.5 | 1.18 |
| Ec fat | 4.5 ± 2.3 | 1.00 | 10.9 ± 0.8 | 1.17 |
| MCC | 4.5 ± 1.2 | — | 9.3 ± 2.7 | — |

The PAI-1 level in hamster plasma was measured by an enzymatic method, which has been used to measure bioactive PAI-1 protein in rat (cell cultures or animal). However, it was reported that this method not only measures PAI-1 activity but also are sensitive to PAI-2. The measured PAI-1 levels in hamster plasma samples of this study are expressed as amidolytic units (AU) per mL. The data show some variation; this is to be expected since the results are obtained on biological, living systems. The data from hamster plasma show a trend that is similar to the data from liver for the PCR analysis of PAI-1. The PAI-1 protein levels are generally lower in the animals of the Treatment Group that were fed a diet containing ethyl cellulose than in the animals of the Control Group that were fed a diet comprising microcrystalline cellulose.

Plasma adiponectin concentrations in hamster samples in this study are listed in Table 7. Compared to the control (MCC) diet, the data show a clear trend. The levels of adiponectin were generally higher for hamsters fed with EC fat and EC dry diet than in the animals of the Control Group that were fed a diet comprising microcrystalline cellulose. The increase in adiponectin protein expression is an important factor in the prevention or treatment of metabolic syndrome.

### % Total Lipids, Triglycerides, Total Cholesterol, and Free Cholesterol in Hamster Livers

The methods for analysis of hamster livers for lipids, triglycerides, free and total cholesterol were summarized as follows. A lyophilized ground liver sample was sandwiched between sand layers in an extraction cell. The cell was placed in the Dionex accelerated solvent extractor and the extraction carried out at 100° C., ˜2000 psig with 75/25 hexane/2-propanol. The sample extract was evaporated to dryness under a nitrogen stream; the residue was brought to constant weight and weighed to determine the % total lipids. The residue was dissolved in a 5/2 v/v chloroform/methanol solution, mixed thoroughly, an aliquot was transferred to a vial, the lipids solubilized in 3% solution of Triton X-100 and the mixture evaporated to dryness under a stream of nitrogen. One mL of deionized water was added to the sample residue, the mixture was mixed thoroughly and the content of triglycerides, free cholesterol and total cholesterol species was determined on a clinical analyzer (as described above for plasma samples). Using the statistical software program JMP the data set was analyzed using multivariate analysis. Outliers were identified based on the Mahalanobis distance for each analyte. Outliers were then omitted from the ANOVA analysis and means testing.

After the hamsters had been fed the diets for three consecutive weeks, the hamsters were sacrificed and the livers were extracted. The liver extracts were analyzed for total % lipids, triglycerides, free cholesterol and total % cholesterol. The levels were measured and determined. The results are listed in Table 8.

TABLE 8

| | | Liver | Liver Free | Liver Total |
|---|---|---|---|---|
| | Liver Total | Triglyceride | Cholesterol | Cholesterol |
| Diet | Lipids (%) | (mg/g) | (mg/g) | (mg/g) |
| EC dry | 12.58 ± 0.74 | 13.35 ± 2.09 | 7.55 ± 0.64 | 8.98 ± 1.15 |
| EC fat | 13.06 ± 0.74 | 13.80 ± 1.69 | 6.78 ± 0.55 | 8.15 ± 1.19 |
| MCC | 20.27 ± 1.47 | 15.77 ± 1.26 | 10.04 ± 1.15 | 42.65 ± 4.58 |

The results indicate that EC fat and EC dry diets showed reductions of 36 and 38%, respectively, in mean total lipids from the control diet MCC. Liver Triglycerides level showed reductions of 12 and 15% for EC fat diet, and EC dry diet, respectively, in mean triglyceride levels from the control diet MCC, respectively. Liver free cholesterol levels showed reductions of 25 and 32% for diets EC dry and EC fat, respectively in mean free cholesterol from the control diet MCC. Liver total cholesterol levels for EC dry and EC fat diets showed reductions of 79 and 81%, respectively, in mean total cholesterol as compared to the control diet, MCC.

Collectively, the results in Example 6 are an indication that water-insoluble cellulose derivatives such as ethyl cellulose are useful for prevention or treatment of metabolic syndrome.

1. A method of preventing or treating metabolic syndrome or a symptom or condition associated with the metabolic syndrome in an individual comprising the step of administering to the individual an effective amount of a water-insoluble cellulose derivative.

2-5. (canceled)

6. The method of claims 1, wherein the water-insoluble cellulose derivative is a water-insoluble cellulose ether.

7. The method of claim 6 wherein the water-insoluble cellulose ether is ethyl cellulose.

8. The method of claims 1 wherein the water-insoluble cellulose derivative is a powder having an average particle size of less than 0.1 millimeter.

9. The method of claims 1, wherein the amount of water-insoluble cellulose derivative administered per day is in the range of from one to ten percent of the total daily weight of the diet of the individual on a dry weight basis.

10. The method of claims 1 wherein the water-insoluble cellulose derivative is administered in combination with a water-soluble cellulose derivative.

11-29. (canceled)

30. A medicament, pharmaceutical composition, food, food ingredient or supplement, or nutraceutical ingredient or supplement comprising ethyl cellulose as an active principle.

31-38. (canceled)
